(19) United States
(12) Patent Application Publication
(10) Pub. No.: US 2008/0303389 A1
(43) Pub. Date: Dec. 11, 2008

(54) SURGICAL CART WITH A MISTER

(76) Inventor: Andy Petrovich, Cumming, GA (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 12/054,167

(22) Filed: Mar. 24, 2008

Related U.S. Application Data
(63) Continuation-in-part of application No. 11/759,761, filed on Jun. 7, 2007.

(51) Int. Cl. A61B 19/02

(52) U.S. Cl. 312/209

(57) ABSTRACT

An improved surgical cart including an enclosure and a misting system at least partially located within the enclosure. The enclosure houses surgical tools and supplies before and after a surgical procedure. The misting system delivers a cleaning solution to the interior of the enclosure to prevent pathological waste from adhering to the surgical tools and supplies.

### CROSS-REFERENCE TO RELATED APPLICATIONS

This application is a Continuation-in-Part of application Ser. No. 11/759,761, entitled SURGICAL CART WITH A MISTER, filed on Jun. 7, 2007, which is incorporated herein by reference in its entirety.

### TECHNICAL FIELD

The present invention relates generally to the field of surgical carts, and more particularly, to an improved surgical cart having a mister.

### BACKGROUND OF THE INVENTION

Supplies and instruments that are used for medical surgery are usually stored in a central supply room that is kept in a sterilized condition. Prior to a particular surgery, the doctor performing the surgery will make a list of the supplies and instruments that will be needed. Immediately preceding the operation, the supplies and instruments are retrieved from the supply room and transported to the operating room where they are set up for the doctor's use. Generally, surgical carts are used to transport the sterile supplies and instruments from the supply room to the operating room, and then transport the used supplies and instruments from the operating room at the conclusion of surgery. At the conclusion of the surgery, the used instruments, supplies, and often pathological waste, is placed in the cart and the cart is sent to a decontamination area or washing station.

In some instances, open surgical carts are used in hospitals or other surgery centers where the supply room is adjacent to the operating room. However, these open carts are rarely used anymore because they allow contamination of the equipment between the supply room and the operating room. Rather, closed surgical carts are typically preferred and are generally standard, as they offer improved protection and sterility. However, it has been found that it is very difficult to clean and sanitize a closed cart because it is difficult to access and clean the interior of the cart.

Regardless of the type of cart that is used, it has also been found that pathological waste (e.g., blood) that is left on the instruments when they are placed in/on the cart quickly dries and becomes very difficult to remove, and can even damage the instruments. For example, certain proteins in blood damage the stainless steel instruments over time, and blood becomes very difficult to remove from a particular instrument once the blood has dried thereon. Additionally, pathological waste often adheres to the cart itself, which can be very difficult and time consuming to remove for the reasons mentioned above. This is especially true with closed carts, as it can be difficult for a user to adequately clean the inside of the cart.

Thus it can be seen that needs exist for improvements to surgical carts that prevent pathological waste from drying on both the cart and the instruments within the cart. It is to the provision of these needs and others that the present invention is primarily directed.

### SUMMARY OF THE INVENTION

The present invention relates to an improved surgical cart including an enclosure and a misting system at least partially located within the enclosure. The enclosure houses surgical tools and supplies before and after a surgical procedure. The misting system delivers a cleaning solution to the interior of the enclosure. An advantage of the surgical cart of the present invention is that the misting system delivers a cleaning solution to the interior of the cart that substantially prevents blood or other pathological waste from drying to and/or corroding any surgical tools contained within the cart. Because blood/pathological waste is not permitted to dry to the tools or interior of the cart, it is much easier to clean and protect the tools/cart from corrosion.

In one aspect, the present invention is a surgical cart having an enclosure for housing surgical tools and supplies before and after a surgical procedure. The surgical cart also includes a misting system that is at least partially located within the enclosure for delivering a cleaning solution to the interior of the enclosure. The misting system includes a pre-pressurized container.

In another aspect, the present invention is a kit for retrofitting a surgical cart. The kit includes at least one pre-pressurized container for delivering a fluid solution into the interior of the cart.

In still other aspect, the present invention is a method of moisturizing the interior of a surgical cart. The method includes locating a pre-pressurized container in proximity to a surgical cart. The pre-pressurized container housing a fluid solution therein. The method also includes activating the pre-pressurized container to disperse the fluid solution within the interior of the surgical cart.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of a known surgical cart.

FIG. 2 is a perspective view of an improved surgical cart according to an exemplary embodiment of the present invention.

FIG. 3 is a front view of the improved surgical cart of FIG. 2.

FIG. 4 is a front view of the improved surgical cart of FIG. 2 shown with the door open for clarity.

FIG. 5 is a side view of the improved surgical cart of FIG. 2.

FIG. 6 shows the components of the misting unit of the improved surgical cart of FIG. 2.

FIG. 7 shows the components of a misting unit according to another exemplary embodiment of the present invention.

FIG. 8 is a perspective view of an improved surgical cart according to another exemplary embodiment of the present invention.

FIG. 9 is a front view of the improved surgical cart of FIG. 8.

FIG. 10 is a front view of the improved surgical cart of FIG. 8 shown with the door open for clarity.

FIG. 11 is a side view of the improved surgical cart of FIG. 8.

FIG. 12 shows the components of a misting unit according to another example embodiment of the present invention.

FIG. 13 is a side view of an improved surgical cart according to still another example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

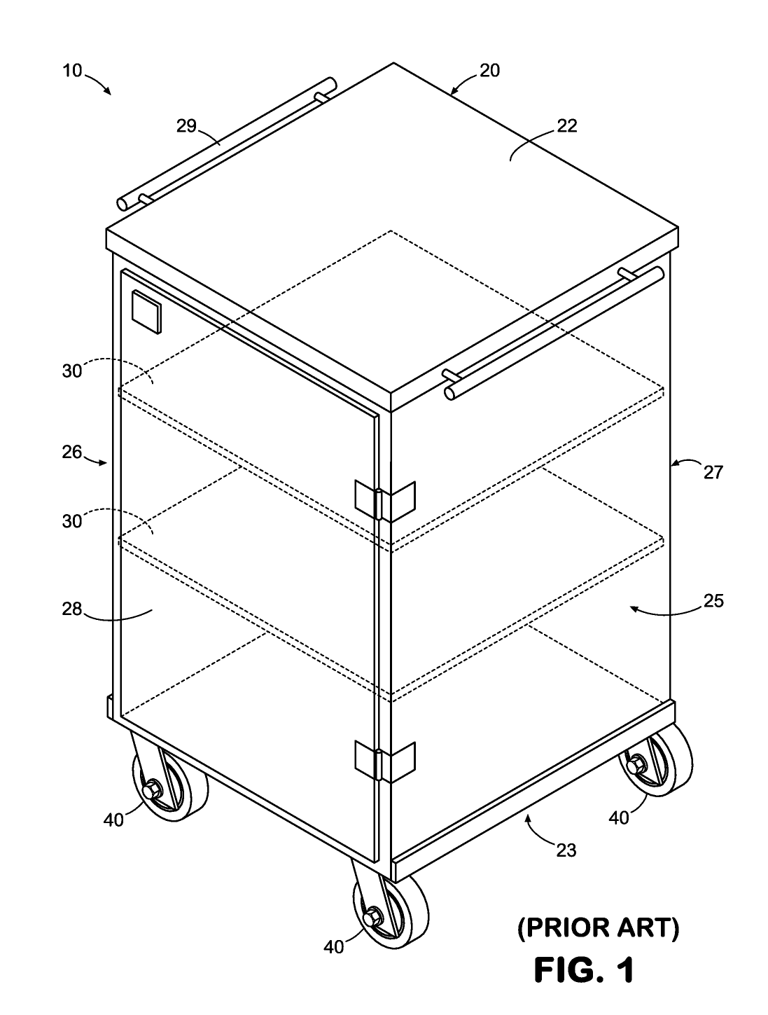

Referring now to the drawing figures, FIG. 1 shows a known closed surgical cart 10. The surgical cart 10 generally includes a rigid enclosure 20 having a top panel 22, bottom panel 23, side panels 25, 26, rear panel 27 and door 28 to permit user access into the interior of the cart. The exterior of the cart 10 can also includes one or more handles 29 to permit a user to grasp and maneuver the cart. One or more racks or shelves 30 can be located within the interior of the cart for receiving a user's surgical instruments, tools, supplies, etc., thereon. Often, these racks 30 are air/fluid permeable, such as wire mesh. Additionally, the cart 10 can also includes a plurality of wheels 40 to ease user transport. Known surgical carts, such as the cart 10 depicted in FIG. 1, are typically constructed from stainless steel.

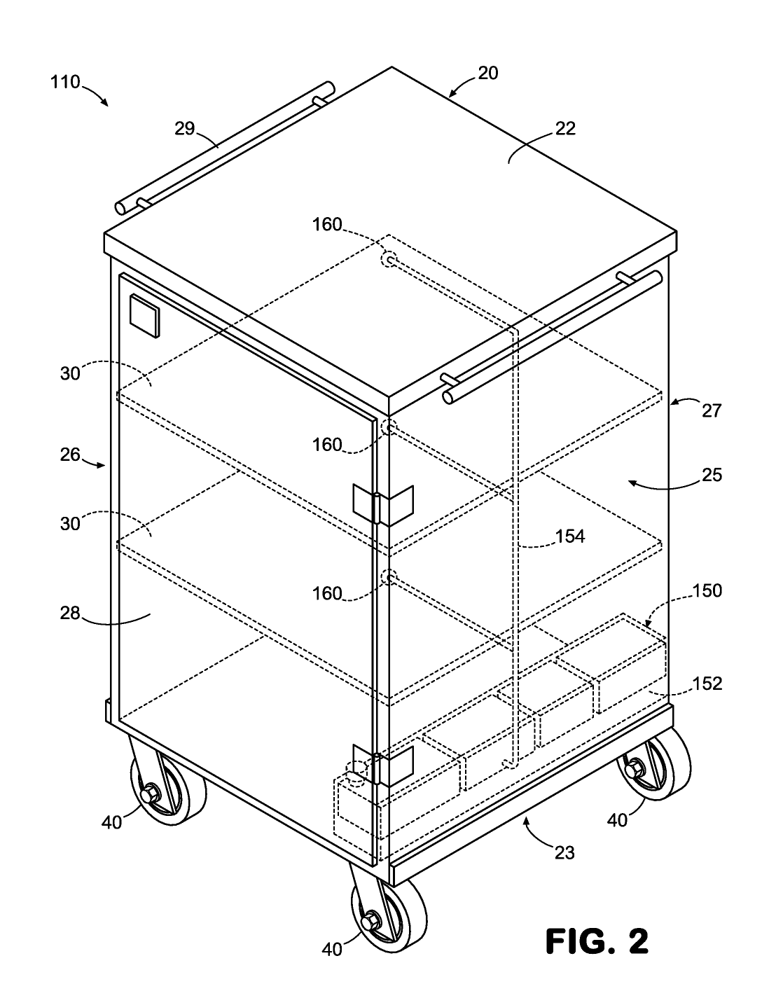

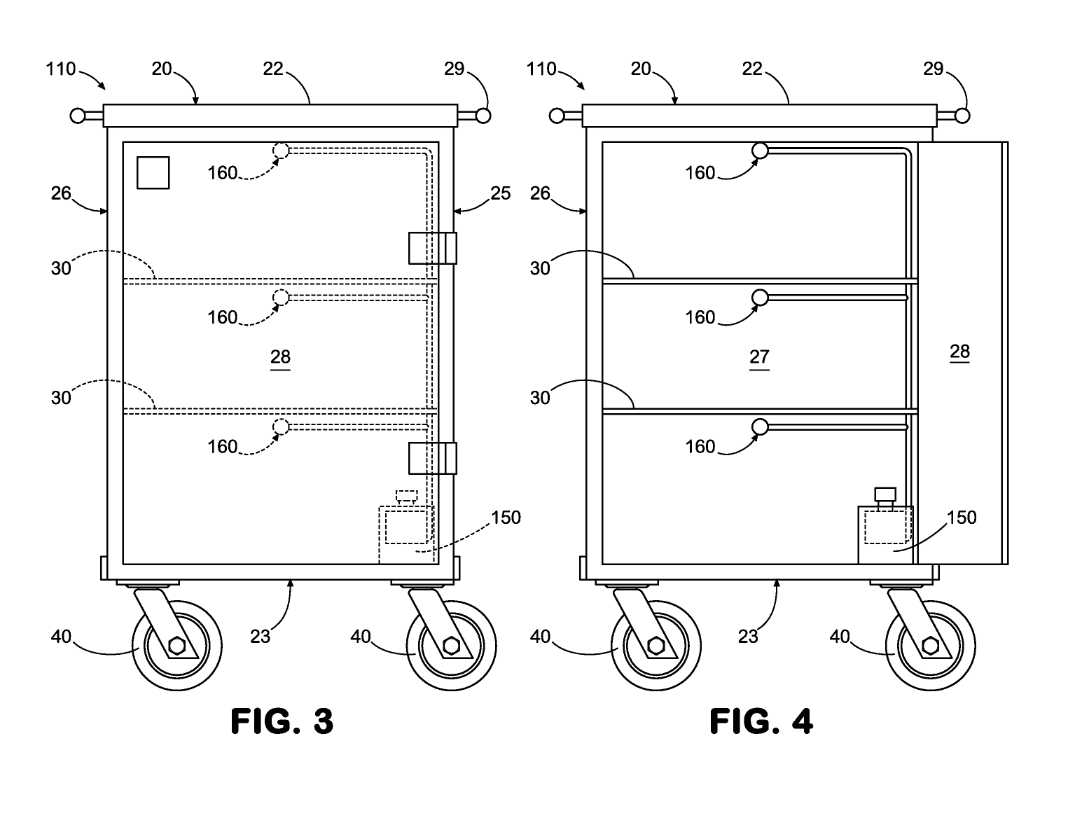

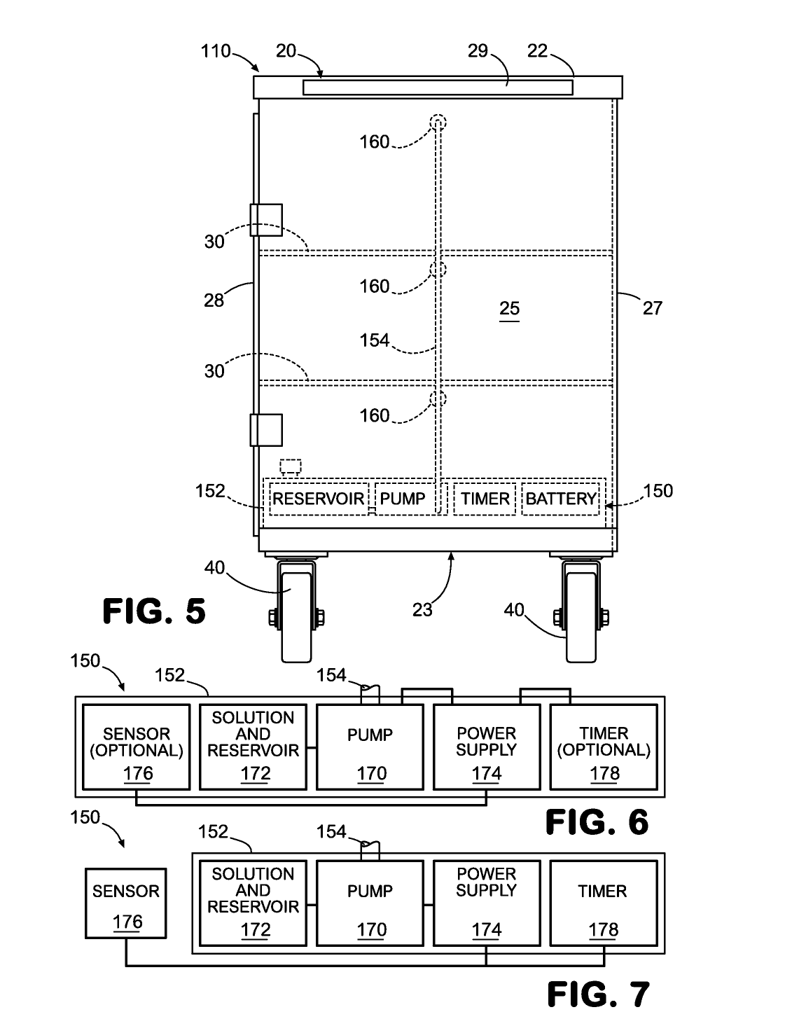

FIGS. 2-5 show an improved surgical cart 110 according to example embodiments of the present invention. In addition to the elements described above, the improved surgical cart 110 includes a misting system 150 to deliver a fluid solution to the inside of the surgical cart. Generally, the misting system 150 can comprise a misting unit 152, piping/tubing 154, and at least one mister 160. The misting unit 152 can further comprise a pump 170, solution reservoir 172, and a power supply 174, as seen in FIGS. 6-7. Optionally, the misting system 150 can also include a door sensor 176 and/or a timer to systematically operate the pump 170.

The misting system 150 can deliver various types of solutions as desired by a user to keep the interior of the cart 110 moist. The moisture provided by the misting system can prevent blood or other pathological waste from drying on any surgical instruments or to the interior surfaces and/or racks 30 of the cart 110. In example embodiments, solutions that can be used with the misting system include water based solutions, enzymatic cleaning solutions, washer concentrate solutions, detergents, lubricant solutions, neutralizers or other types of cleaning solutions as desired. In commercial embodiments, it has been found that enzymatic cleaning solutions are highly effective in prohibiting pathological waste from drying to, corroding, or damaging the instruments and/or cart 110. The solution can be contained within the solution reservoir 172 and can be refilled by a user when needed. Alternatively, the solution reservoir can comprise a refillable/replaceable cartridge that can be removed from the misting unit 152 and replaced therein as desired.

The misting unit 152 can be any shape as preferred by a user. In exemplary embodiments, the misting unit 152 is an elongated cube or rectangular box. The unit 152 can contain the pump 170, solution reservoir 172, and power supply 174 and can also provide user access to the internal components therein, such as the power supply, solution reservoir, etc. The exterior of the misting unit 152 can be constructed from stainless steel, steel, aluminum, rubber, plastic, or other materials as desired by a user. However, in commercial embodiments, stainless steel is preferred because of its properties as a long-lasting, easy to clean, and durable material. The misting unit 152 can be positioned in any one of a multitude of possible locations, such as, within the interior of the cart 110, as shown in the drawing figures, in proximity to the exterior of the cart, or in other suitable locations as desired by a user. In commercial embodiments, the misting unit 152 is preferably stationed in the interior of the cart 110, wherein the bottom panel 23 supports the unit. Optionally, the misting unit 152 can also rest against a side panel 25, 26 or rear panel 27 so that the unit is out of the user's way as much as possible. In alternative embodiments, the misting unit 152 can hang from the interior top panel 22 of the cart 110. In still other embodiments, the pump 170, solution reservoir 172, power supply 174, and optionally the sensor 176 and timer 178, are not contained within a defined misting unit, but rather are individually located within or adjacent to the surgical cart 110.

The components of the misting unit 152 can be energized by the power supply 174. The power supply 174 can comprise a conventional DC power source, such as alkaline type batteries, rechargeable batteries, lithium ion, nickel-cadmium, nickel-metal-hydride, button batteries, or any other suitable power source. In alternative embodiments, the power supply 174 comprises rechargeable batteries that are charged by removing the power supply from the misting unit 152 and placing the power supply in a base recharger when needed. In such exemplary embodiments, it is preferable that the misting unit 152 provides user access to the power supply 174 to permit a user to remove the same for the purpose of recharging. In other alternative embodiments, power to the misting unit 152 is derived from AC power, such as plugging the unit into an electrical outlet. Additionally, the power supply 174 can further include an on/off switch for user-controlled operation. In still other embodiments, the pump 170 can be gravity fed, hand operated, or pre-pressurized, such that no electrical power supply 174 is required.

The pump 170 of the present invention can be any fluid pump as desired by a user capable of transporting a liquid solution from the solution reservoir 172 to at least one mister 160. The pump 170 can comprise electric pumps, gravity fed systems, hand operated pumps, pre-pressurized pumps, and other pumps as desired. The solution reservoir 172 can contain the cleaning solution until received by the pump 170, and the capacity of the reservoir can vary with the size of the surgical cart 110 and/or as desired by a user. In alternative embodiments, the misting unit 152 includes a door sensor 176 coupled to the power supply 174 for permitting the power supply to energize the pump only when the door 28 of the cart 110 is closed. In other embodiments, the sensor 176 can be remotely located in regards to the misting unit 152 or can be located adjacent to the exterior of the unit, between the unit and the door 28, or as otherwise determined by a particular user. In still other embodiments, the unit 152 can include a timer 178 for selectively operating the pump at preset or user determined intervals.

Generally, one or more sections of tubing 154 can be coupled to the pump 170 of the misting unit 152 for transporting the cleaning solution from the unit and/or the solution reservoir 172 to the mister(s) 160. The tubing 154 can be constructed from stainless steel, steel, aluminum, rubber, plastic, or other materials as desired by a user. Commercial embodiments of the present invention use stainless steel tubing 154, for similar reasons as described above in relation to the material properties of the misting unit 152. The tubing 154 can be welded, bonded, latched or otherwise secured to and/or supported by the interior of the cart 110. In alternative embodiments, a mister 160 can be directly connected to the misting unit 152 without the use of any tubing/piping. Such embodiments are especially useful when the misting unit 152 is supported beneath the interior top panel 22 and the surgical cart 110 has no interior racks 30, or the racks are air/fluid permeable or comprised of wire mesh.

In exemplary embodiments, each section of tubing 154 can have at least one mister 160 coupled thereto for producing a fine mist within the surgical cart 110. In alternate embodiments, the mister(s) can produce a spray, jet, wash, droplets, or other methods of delivering a liquid as desired to the interior of the cart 110. The mister(s) can comprise nozzles, sprinklers, atomizers, and/or conventional mist generators. The mister(s) 160 can produce a preset level of mist/moisture, or the mister(s) can be modified by the user to produce more or less mist/moisture. In commercial embodiments of the present invention at least one mister 160 is provided for each rack 30, so that each rack receives a direct supply of moisture to prevent pathological waste from binding to or corroding the surgical instruments. Optionally, more than one mister 160 can be utilized for each rack 30. In alternative embodiments, only one mister 160 is provided for the surgical cart 110.

In operation, once a user has completed using a particular surgical tool or instrument, the user can place the instruments into the surgical cart 110. Often these instruments are covered in blood or other pathological waste. Once the first tool or instrument is placed in the cart 110 a user can begin operation of the misting system 150 by turning the power supply 174 “on”. If a particular cart 110 is equipped with a door sensor 176, the misting system 150 will not begin to operate until the door 28 has been closed. Once the misting system 150 has become operational, the pump 170 will begin to transport the cleaning solution from the solution reservoir 172 through the tubing 154 (if equipped with such) and ultimately to at least one mister 160. Once the cleaning solution has reached the mister 160 the solution is dispensed into the cart 110 in the form of a mist, spray, wash, etc. to moisturize the interior of the cart and the contents therein. If the cart 110 is equipped with a timer 178, the timer can selectively regulate operational intervals and the duration of the same. By moisturizing the interior of the cart 110, blood and other pathological wasted is prohibited from drying to and/or corroding the instruments or cart. Among other factors, the amount of moisture that is needed can vary with the size of the cart 110, number of misters 160, type of cleaning solution used, and duration of misting system 150 operation. In alternative embodiments, the cart 110 can comprise a moisture collection pan/reservoir and/or a drain plug to aid in moisture removal and clean-up. In still other alternative embodiments, the pump 170 can recirculate the cleaning solution once the reservoir has been emptied.

In additional commercial embodiments of the present invention the misting system 150 can be produced and sold as a retrofit kit. The retrofit kit can allow a user to modify an existing surgical cart, such that the retrofitted cart is capable of delivering a cleaning mist to the interior of the cart. The kit can include a pump 170, solution reservoir 172, power supply 174, and optionally a sensor 176 and/or timer 178. In alternative embodiments, the kit can include a gravity fed, hand operated, or pre-pressurized pump instead of a pump/power supply combination. The kit can also include at least one mister 160 and at least one section of tubing 154 to channel the solution between the pump and at least one mister.

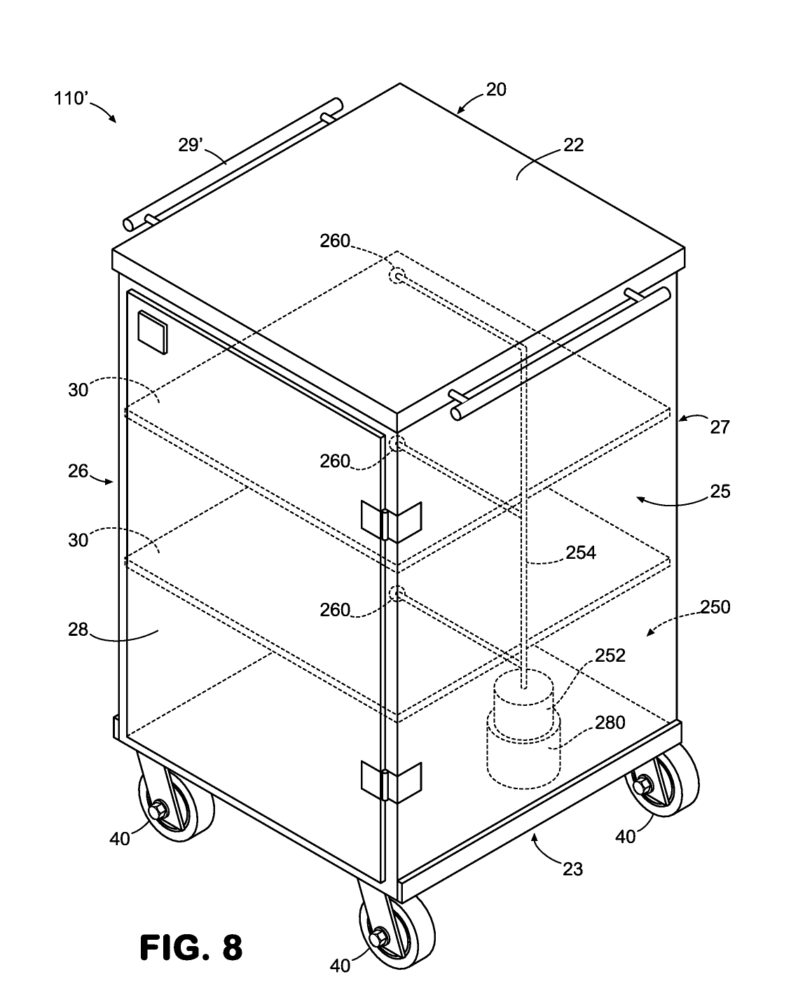

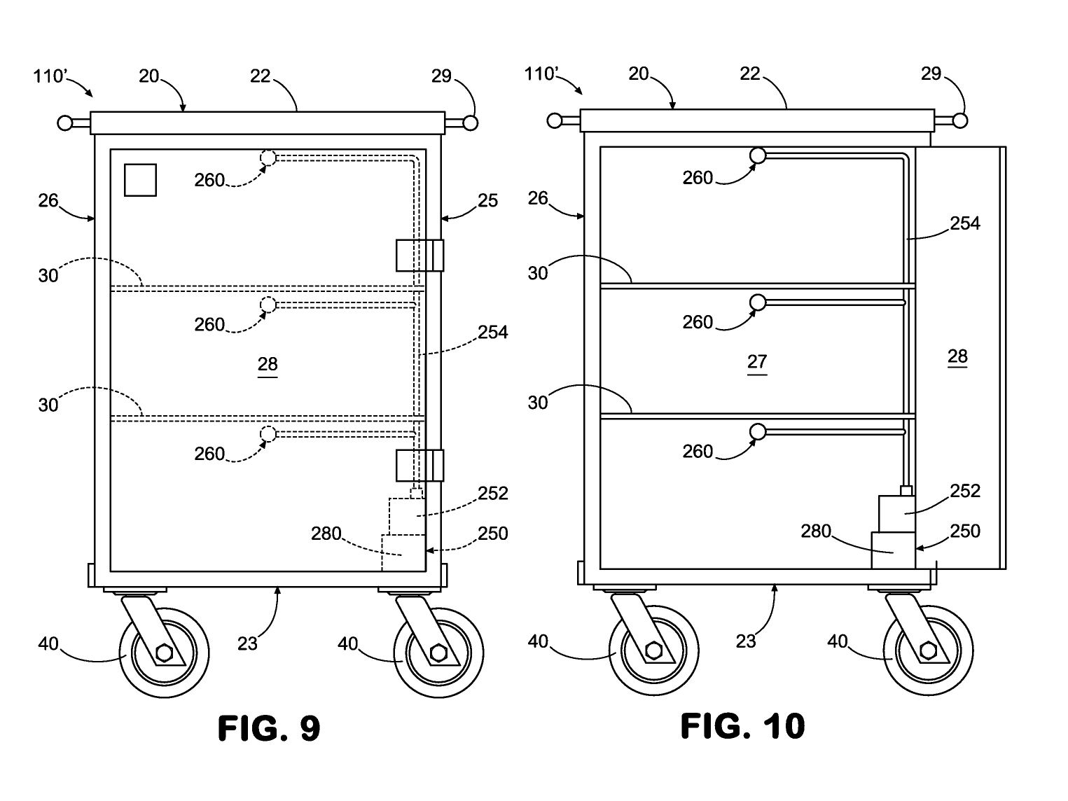

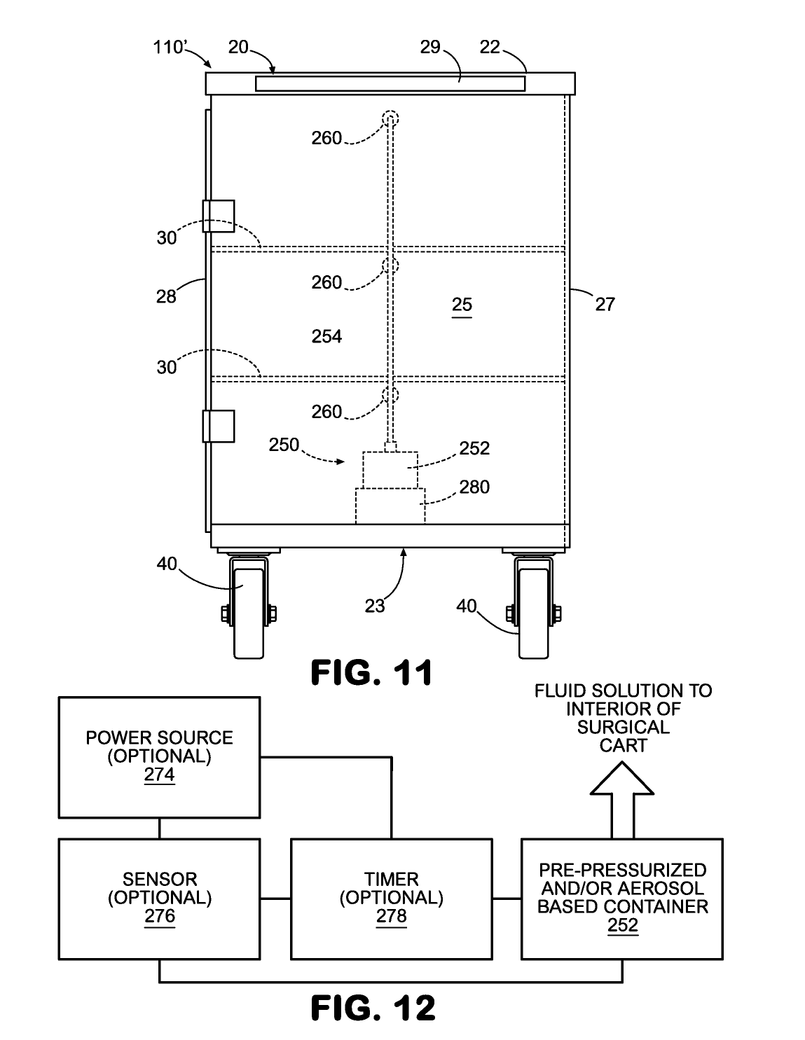

In other example embodiments of the present invention, an improved surgical cart 110′ is shown in FIGS. 8-11. The cart 110′ includes a misting system 250 that delivers moisture to the interior of the cart. In example embodiments the misting system 250 comprises at least one pre-pressurized container 252 for storing a cleaning solution/fluid therein and releasing the same into the interior of the cart 110′. In other example embodiments, the misting system 250 further comprises at least one section of piping or tubing 254, and at least one mister 260 for delivering the fluid to the interior of the cart 110′. In still other example embodiments, as seen in FIG. 12, the misting system 250 can include at least one sensor 276, at least one timer 278, and/or a container mount 280 (container mount shown in FIGS. 8-11) as desired. Additionally, the misting system can include a power source 274, such as a battery or other power device. Similar to previous embodiments, the misting system 250 can deliver various types of fluid solutions to keep the interior of the cart 110′ moist, such as water based solutions, enzymatic cleaning solutions, washer concentrate solutions, detergents, lubricant solutions, neutralizers, etc. Optionally, color (such as food coloring) can be added to the fluid so that it can easily be determined by a user whether the pre-pressurized container 252 has been discharged or not. Still optionally, a foaming solution can be added to the pre-pressurized container 252 to indicate the same and/or to improve the efficiency of the fluid solution. Different types and concentrations of fluid solutions can be added to the pre-pressurized containers 252 as desired by a user. In preferred example embodiments, the pre-pressurized container 252 is an aerosol based container, wherein the container contains a pressurized fluid solution. Alternatively, other types of pre-pressurized containers/pumps can be utilized, such as atomizers, gravity fed, and/or hand operated pumps. In still other example embodiments, the container 252 is refillable.

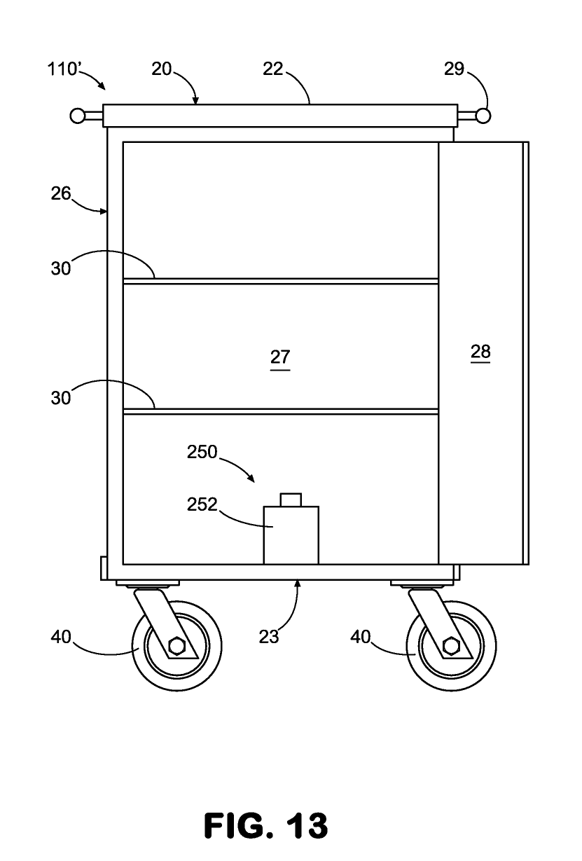

Referring again to FIGS. 8-11, in example embodiments the pre-pressurized pump 252 can be coupled to at least one section of piping or tubing 254, and the tubing can be coupled to at least one mister 260. In alternate example embodiments, two or more misters 260 can be used with the misting system 250. In still other embodiments, the misting system 250 comprises only a pre-pressurized container 252, similar to a “bug bomb” that a user “activates” and allows the contents of the container 252 to disperse within the cart 110′ (as seen in FIG. 13). The pre-pressurized container 252 can optionally have a mister 260 directly coupled to the container (such as a spray nozzle). As shown in FIG. 8, the pre-pressurized container 252 can engage a corresponding container mount 280 and can be secured thereto/therein to substantially prevent the container 252 from moving around within the cart 110′. The container mount 280 may be permanently or releasably coupled to an interior or exterior face of the cart 110′. For example, the mount 280 can be coupled to the ceiling 22, sides 25, 26, rear 27, bottom 23, or door 28 of the cart 110′ as desired. The container mount 280 can be coupled to the cart 110′ with epoxy, glue, silicone, hook and loop fasteners, bolts, screws, magnets, or other conventional fasteners. The mount 280 can secure the pre-pressurized container 252 therein/thereto with mechanical fasteners, straps, hooks, magnets, nesting, or otherwise secure the same. In alternate example embodiments, the pre-pressurized container 252 can be secured to the interior or exterior of the cart 110′ directly, such that a container mount 280 is not utilized. In such embodiments, the container 252 can be coupled to the cart 110′ with epoxy, glue, silicone, hook and loop fasteners, bolts, screws, magnets, or other conventional fasteners. In still other embodiments, the pre-pressurized container is not secured to the cart 110′.

As previously discussed, example embodiments of the present invention can include a sensor 276 and/or a timer 278 that are mechanically and/or electrically coupled to the pre-pressurized container 252 for intermittently releasing the fluid contents of the container. The sensor 276 and/or timer 278 can be battery powered or can be plugged into an electrical outlet as desired. The cart 110′ of the present invention can also include one or more strips of material within the interior and/or exterior of the cart that change color or otherwise alert a user that the pre-pressurized container 252 has been discharged. In alternate embodiments, the pre-pressurized container 252 can include a nozzle and/or a strip of material that alerts a user the container has been discharged.

The present invention also includes a retrofit kit that allows a user to retrofit a pre-existing surgical cart with the misting system 250 that includes at least one pre-pressurized container 252. The pre-pressurized container 252 can be used within a pre-existing surgical cart to keep the interior of the cart moist as desired. The kit can optionally include at least one container mount 280 for securing the container 252 within/in proximity to the cart, at least one sensor 276, at least one timer 278, at least one section of tubing 254, and/or at least one mister. Alternatively, the kit can just include a single pre-pressurized container 252 that can be located within a surgical cart as desired by user. In still other embodiments, the kit can include two or more pre-pressurized containers 252.

While the invention has been described with reference to preferred and example embodiments, it will be understood by those skilled in the art that a variety of modifications, additions and deletions are within the scope of the invention, as defined by the following claims.

What is claimed is:

1. A surgical cart comprising:
an enclosure for housing surgical tools and supplies before and after a surgical procedure; and
a misting system at least partially located within the enclosure for delivering a cleaning solution to the interior of the enclosure;
wherein the misting system comprises a pre-pressurized container.

2. The surgical cart of claim 1, wherein the system further comprises at least one mister coupled to the pre-pressurized container.

3. The surgical cart of claim 2, wherein the system further comprises at least one section of tubing for channeling a fluid solution from the pre-pressurized container to the at least one mister.

4. The surgical cart of claim 1, wherein the pre-pressurized container is an aerosol based container.

5. The surgical cart of claim 1, wherein the cart further comprises a container mount for securing the pre-pressurized container to the cart.

6. A kit for retrofitting a surgical cart, the kit comprising:
at least one pre-pressurized container for delivering a fluid solution into the interior of the cart.

7. The kit of claim 6, wherein the kit further comprises a container mount for securing the at least one pre-pressurized container to the cart.

8. The kit of claim 6, wherein the kit further comprises at least one mister coupled to the pre-pressurized container.

9. The kit of claim 6, wherein the pre-pressurized container is an aerosol based container.

10. The kit of claim 6, wherein the kit further comprises two pre-pressurized containers.

11. A method of moisturizing the interior of a surgical cart comprising:
locating a pre-pressurized container in proximity to a surgical cart, the pre-pressurized container housing a fluid solution therein; and
activating the pre-pressurized container to disperse the fluid solution within the interior of the surgical cart.
